(19) United States
(12) Patent Application Publication
(10) Pub. No.: US 2008/0004279 A1
(43) Pub. Date: Jan. 3, 2008

(54) Use of C-Kit Inhibitors for Treating Plasmodium Related Diseases

(76) Inventors: Alain Moussy, Paris (FR); Jean-Pierre Kinet, Lexington, MA (US)

Correspondence Address: FOLEY AND LARDNER LLP;SUITE 500, 3000 K STREET NW, WASHINGTON, DC, 20007, US

(21) Appl. No.: 11/587,271

(22) PCT Filed: Apr. 19, 2005
(86) PCT No.: PCT/IB05/01390

§ 371 Date: Oct. 23, 2006

Related U.S. Application Data
(60) Provisional application No. 60/564,599, filed on Apr. 23, 2004.

(51) Int. Cl. A61K 31/4436; A61K 31/443; A61K 31/497; A61K 31/517; A61K 31/5377; A61K 35/00; A61P 33/00; A61P 33/02; A61P 33/06

(52) U.S. Cl. 514/236.8; 514/253.1; 514/266.1; 514/342; 514/789

(57) ABSTRACT

The present invention relates to a method for treating plasmodium related diseases comprising administering a compound capable of inhibiting tyrosine kinases, to a human in need of such treatment. Such compounds can be chosen from tyrosine kinase inhibitors including c-kit inhibitors and more particularly non-toxic, selective and potent tyrosine kinases inhibitors. Preferably, said inhibitor is unable to promote death of IL-3 dependent cells cultured in presence of IL-3.

The present invention relates to a method for treating plasmodium related diseases comprising administering a compound capable of inhibiting tyrosine kinases, to a human in need of such treatment. Such compounds can be chosen from tyrosine kinase inhibitors including c-kit inhibitors and more particularly non-toxic, selective and potent tyrosine kinases inhibitors; Preferably, said inhibitor is unable to promote death of IL-3 dependent cells cultured in presence of IL-3.

Malaria is a devastating disease caused by a unicellular protozoan, Plasmodium, which affects 3.7 million people every year. Resistance of the parasite to classical treatments such as chloroquine requires the development of new drugs (Gazarini, 2003). Plasmodium, the causative agent of malaria, must first infect hepatocytes to initiate a mammalian infection. Sporozoites migrate through several hepatocytes, by reaching their plasma membranes, before infection is finally established in one of them (Carolo, 2003).

Tha pathogenicity of Plasmodium is due to the unique ability of infected erythrocytes (IRBCs) to adhere to vascular endothelium (Yipp, 2003, Blood). Src-family kinase signalling has been shown to modulate the adhesion of Plasmodium falciparum on human microvascular endothelium under flow. Furthermore, Src-family kinase activity was also required for cytoadherence to intact human microvessels in a human/severe combined immunodeficient (SCID) mouse model in vivo.

Protein tyrosine kinase activity was found to be distributed in all the stages of P. falciparum parasite maturation. Membrane bound PTK activity was found to be increased during maturation process (ring stage to trophozoite stage) in chloroquine sensitive strains. In vivo conversion of the schizont stage to ring stage via release of merozoites was associated with a decrease in PTK activity. Chloroquine inhibited the membrane bound PTK activity in a dose dependent manner (IC50=45 microM). Kinetic studies show that chloroquine is a competitive inhibitor of PTK with respect to peptide substrate but a noncompetitive with respect to ATP indicating that chloroquine inhibits PTK activity by binding with protein substrate binding site.

To gain insight into the mechanisms that control Plasmodium cell cycle, the effects of kinase inhibitors genistein (200 microM), staurosporine (1 microM), R03 (1 microM), and tyrphostins B44 (100 microM) on the blood-stage cycle of the rodent malaria parasite, Plasmodium chabaudi, has been evaluated (Gazarini, Braz J Med Biol Res. November 2003; 36(11):1465-9). In vitro studies showed that kinase inhibitors retard or prevent maturation of the intraerythrocytic parasites. The results of the studies support the hypothesis that the maturation of the intraerythrocytic cycle of malaria parasites requires phosphorylation. In this respect, Gazarini recently reported a high Ca2+ microenvironment surrounding the parasite within red blood cells. Several kinase activities are modulated by Ca2+ (Gazarini, 2003).

Glycosylphosphatidylinositol (GPI) is a major toxin of Plasmodium falciparum origin responsible for nitric oxide (NO) production in host cells. Purified malarial GPI is sufficient to induce NO release in a time- and dose-dependent manner in macrophages and vascular endothelial cells, and regulates inducible NO synthase expression in macrophages. GPI-induced NO production was blocked by the NO synthase-specific inhibitor L-N-monomethylarginine. GPI also synergizes with IFN-gamma in regulating NO production. The structurally related molecules dipalmitoylphosphatidylinositol and iM4 glycoinositolphospholipid from Leishmania mexicana had no such activity, and the latter antagonized IFN-gamma-induced NO output. GPI activates macrophages by initiating an early onset tyrosine kinase-mediated signaling process, similar to that induced by total parasite extracts. The tyrosine kinase antagonists tyrphostin and genistein inhibited the release of NO by parasite extracts and by GPI, alone or in combination with IFN-gamma, demonstrating the involvement of one or more tyrosine kinases in the signaling cascade. GPI-induced NO release was also blocked by the protein kinase C inhibitor calphostin C, demonstrating a role for protein kinase C in GPI-mediated cell signaling, and by pyrrolidine dithiocarbamate, indicating the involvement of the NF-kappa B/c-rel family of transcription factors in cell activation. A neutralizing mAb to malarial GPI inhibited NO production induced by GPI and total malarial parasite extracts in human vascular endothelial cells and murine macrophages, indicating that GPI is a necessary agent of parasite origin in parasite-induced NO output. Thus, in contrast to dipalmitoylphosphatidylinositol and glycoinositolphospholipids of Leishmania, malarial GPI initiates a protein tyrosine kinase- and protein kinase C-mediated signal transduction pathway, regulating inducible NO synthase expression with the participation of NF-kappa B/c-rel, which leads to macrophage and vascular endothelial cell activation and downstream production of NO. These events may play a role in the etiology of severe malaria.

Furthermore, the glycosylphosphatidylinositols (GPIs) of Plasmodium falciparum are believed to contribute to the pathogenesis of malaria by inducing the secretion of proinflammatory cytokines by macrophages. Previous studies have shown that P. falciparum GPIs elicit toxic immune responses by protein tyrosine kinase (PTK) and protein kinase C (PKC)-mediated cell signaling pathways, which are activated by the carbohydrate and acyl moieties of the intact GPIs, respectively. The induction of TNF-alpha by P. falciparum GPIs in macrophages is mediated by the recognition of the distal fourth mannose residue. This event is critical but not sufficient for the productive cell signaling; interaction by the acylglycerol moiety of GPIs is also required. These novel interactions are coupled to previously demonstrated PTK and PKC pathways, since the specific inhibitors of these kinases effectively blocked the GPI-induced TNF-alpha production (Vijaykumar, J Biol Chem. Mar. 9, 2001;276(10):6909-12. Epub Jan. 10, 2001).

Gastrointestinal nematode infection has been shown to be associated with mucosal mast cell (MMC) hyperplasia. In the mouse, this is accompanied by the release of substantial quantities of the chymase mouse mast cell proteinase-1 (mMCP-1) into the gut lumen and peripheral bloodstream. (Pemberton A D, Brown J K, Wright S H, Knight P A, McPhee M L, McEuen A R, Forse P A, Miller H R. Purification and characterization of mouse mast cell proteinase-2 and the differential expression and release of mouse mast cell proteinase-1 and -2 in vivo. Clin Exp Allergy. July 2003 ;33(7):1005-12).

In connection with the present invention, it has been found for the first time that tyrosine kinase inhibitors and in particular c-kit inhibitors could be a new route for treating plasmodium related diseases through inhibition of the growth of plasmodium falciparum.

### DESCRIPTION

The present invention relates to a method for treating and/or preventing or delaying infections with plasmodium or nematodes comprising administering a tyrosine kinase inhibitor to a human in need of such treatment.

Said method for treating plasmodium or nematodes related diseases can comprise administering a c-kit inhibitor to a human in need of such treatment.

Preferred compounds are c-kit inhibitor, more particularly a non-toxic, selective and potent c-kit inhibitor. Such inhibitors can be selected from the group consisting of 2-(3-Substitutedaryl)amino-4-aryl-thiazoles such as 2-(3-amino)arylamino-4-aryl-thiazoles, 2-aminoaryloxazoles, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating plasmodium related diseases comprising administering a non toxic, potent and selective c-kit inhibitor is a pyrimidine derivatives, more particularly N-phenyl-2-pyrimidine-amine derivatives of formula I:

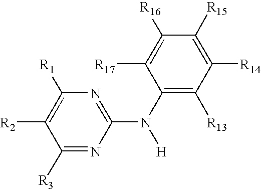

wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II:

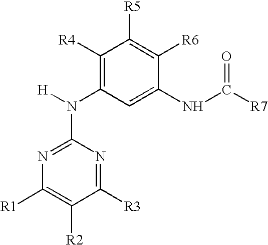

Wherein R1, R2 and R3 are independently chosen from H, F, Cl, Br, I, a C1-C5 alkyl or a cyclic or heterocyclic group, especially a pyridyl group;

R4, R5 and R6 are independently chosen from H, F, Cl, Br, I, a C1-C5 alkyl, especially a methyl group;

and R7 is a phenyl group bearing at least one substituent, which in turn possesses at least one basic site, such as an amino function.

Preferably, R7 is the following group:

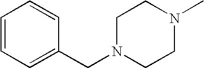

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one basic site, such as an amino function, for example the group:

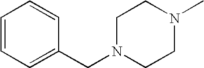

Therefore, in a preferred embodiment, the invention relates to a method for treating plasmodium related diseases comprising the administration of an effective amount of the compound known in the art as CGP57148B:

4-(4-méhylpipérazine-1-ylméthyl)-N-[4-méthyl-3-(4-pyridine-3-yl)pyrimidine-2ylamino)phényl]-benzamide corresponding to the following formula:

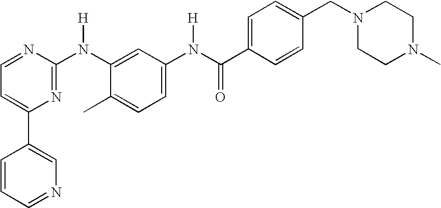

The preparation of this compound is described in example 21 of EP 564 409 and the β-form, which is particularly useful is described in WO 99/03854.

In another preferred embodiment, the invention contemplates the method mentioned above, wherein said c-kit inhibitor is selected from 2-(3-Substitutedaryl)amino-4-aryl-thiazoles such as those for which the applicant filed PCT/IB2005/000401, incorporated herein by reference, especially compounds of formula III:

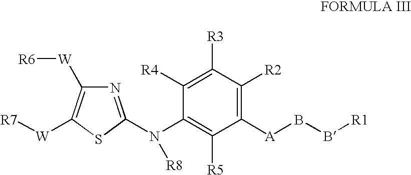

wherein

R⁶ and R⁷ are independently from each other chosen from one of the following:

i) hydrogen, a halogen (selected from F, Cl, Br or I),

ii) an alkyl¹ group defined as a linear, branched or cycloalkyl group containing from 1 to 10 carbon atoms, or from 2 or 3 to 10 carbon atoms, (for example methyl, ethyl, propyl, butyl, pentyl, hexyl . . . ) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl;

(iii) an aryl¹ group defined as phenyl or a substituted variant thereof bearing any combination, at any one ring position, of one or more substituents such as
halogen(selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;

(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;

(v) trifluoromethyl, carboxyl, cyano, nitro, formyl, hydroxy, N(alkyl¹)(alkyl¹), and amino, the latter nitrogen substituents optionally in the form of a basic nitrogen functionality.

R⁸ is one of the following:

(i) hydrogen, or

(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or

(iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R2, R3, R4 and R5 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or ), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

A is: CH2; O, S, SO2, CO, or COO,

B is a bond or NH, NCH3, NR*, (CH2)n (n is 0, 1 or 2), O, S, SO2, CO, or COO,

B′ is a bond or NH, NCH3, NR*, (CH2)n (n is 0, 1 or 2), O, S, SO2, CO or COO;

R* being an alkyl¹, aryl¹ or heteroaryl¹

W is a bond or a linker selected from NH, NHCO, NHCOO, NHCONH, NHSO2, NHSO2NH, CO, CONH, COO, COCH2, (CH2)n (n is 0, 1 or 2), CH2-CO, CH2COO, CH2-NH, O, OCH2, S, SO2, and SO2NH

R¹ is:

a) a linear or branched alkyl group containing from 1 to 10 carbon atoms optionally substituted with at least one heteroatom, notably a halogen selected from I, Cl, Br and F, and/or bearing a pendant basic nitrogen functionality;

b) an aryl or heteroaryl group optionally substituted by an alkyl or aryl group optionally substituted with a heteroatom, notably a halogen selected from I, Cl, Br and F or bearing a pendant basic nitrogen functionality

c) an alkyl¹, aryl¹ or heteroaryl¹.

It will be understood that a C₁-C10 alkyl encompasses a methyl, ethyl, propyl, and a C2 to C4 alkyl or a C2 to C10 alkyl.

For example, a subset of compounds may correspond to

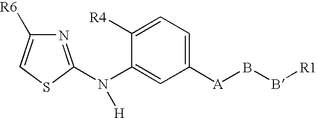

Wherein R1, R4 and R6 have the meaning as defined above.

It will be understood that A-B-B′ includes but is not limited to:

CH2, CH2-CO, CH2-CO—CH2, CH2COO, CH2-CH2-CO, CH2-CH2-COO, CH2-NH, CH2-CH2-NH, CH2-NH—CH2 or CH2-NH—CO or CH2-CO—NH

It will be understood that A-B-B′ also includes but is not limited to:

CO—CH2, COO—CH2, CO—CH2-CH2, CO—NH, or CO—NH—CH2 as well as O—CH2

It will also be understood that NH in B or B′ can also be NCH3

In the above formula III, when W is other than a single bond, it will be understood that A can be also be NH or NCH3.

In the above formula, the following combinations are contemplated:
R6 is (iv), R4 is H or CH3, A-B—B′ is CO—NH and R1 is as defined above.
R6 is (iv), R4 is H or CH3, A-B—B′ is CH2-CO—NH and R1 is as defined above.
R6 is (iv), R4 is H or CH3, A-B—B′ is CH2-CO and R1 is as defined above.
R6 is (iv), R4 is H or CH3, A-B—B′ is CH2-NH—CO and R1 is as defined above.
R6 is (iv), R4 is H or CH3, A-B—B′ is CH2-NH and R1 is as defined above.
R6 is (iv), R4 is H or CH3, A-B—B′ is CH2 and R1 is as defined above.
R6 is W-(iv), R4 is a C1-C2 alkyl, A-B—B′ is CO—NH and R1 is as defined above.
R6 is (iv), R4 is a C1-C2 alkyl, A-B—B′ is CH2-CO—NH and R1 is as defined above.
R6 is (iv), R4 is a C1-C2 alkyl, A-B—B′ is CH2-CO and R1 is as defined above.
R6 is a pyridyl according to (iv), R4 is a C1-C2 alkyl, A-B—B′ is CO—NH, CH2-CO—NH, CH2-CO, CH2-NH, CH2-NH—CO and R1 is as defined above.

In the above combination, R1 can be an alkyl¹.

In the above combination, R1 can be an aryl¹.

In the above combination, R1 can be an heteroaryl¹.

In another preferred embodiment, the invention contemplated the method mentioned above, wherein said c-kit inhibitor is selected from 2-(3-amino)arylamino-4-aryl-thiazoles such as those for which the applicant filed WO 2004/014903, incorporated herein in the description, especially compounds of formula IV:

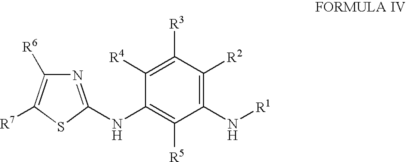

and wherein R¹ is:

a) a linear or branched alkyl group containing from 1 to 10 carbon atoms optionally substituted with at least one heteroatom, notably a halogen selected from I, Cl, Br and F, and/or bearing a pendant basic nitrogen functionality;

b) an aryl or heteroaryl group optionally substituted by an alkyl or aryl group optionally substituted with a heteroatom, notably a halogen selected from I, Cl, Br and F or bearing a pendant basic nitrogen functionality;

c) a —CO—NH—R, —CO—R, —CO—OR or a —CO—NRR′ group, wherein R and R′ are independently chosen from H or an aryl, heteroaryl, alkyl and cycloalkyl group optionally substituted with at least one heteroatom, notably a halogen selected from I, Cl, Br and F, and/or bearing a pendant basic nitrogen functionality;

R² is hydrogen, halogen or a linear or branched alkyl group containing from 1 to 10 carbon atoms, trifluoromethyl or alkoxy;

R³ is hydrogen, halogen or a linear or branched alkyl group containing from 1 to 10 carbon atoms, trifluoromethyl or alkoxy;

R⁴ is hydrogen, halogen or a linear or branched alkyl group containing from 1 to 10 carbon atoms, trifluoromethyl or alkoxy;

R⁵ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality;

and R⁷ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another preferred embodiment, when R¹ has the meaning depicted in c) above, the invention is directed to compounds of the following formulas:

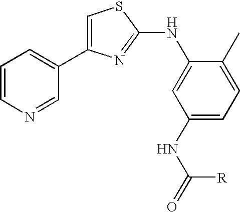

wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted by an alkyl, a cycloalkyl, an aryl or heteroaryl group optionally substituted with a heteroatom, notably a halogen selected from I, Cl, Br and F and/or bearing a pendant basic nitrogen functionality.

Among the particular compounds in which R1 has the meaning as depicted in c) above, the invention is directed to amide-aniline, amide-benzylamine, amide-phenol, urea compounds of the following formulas respectively:

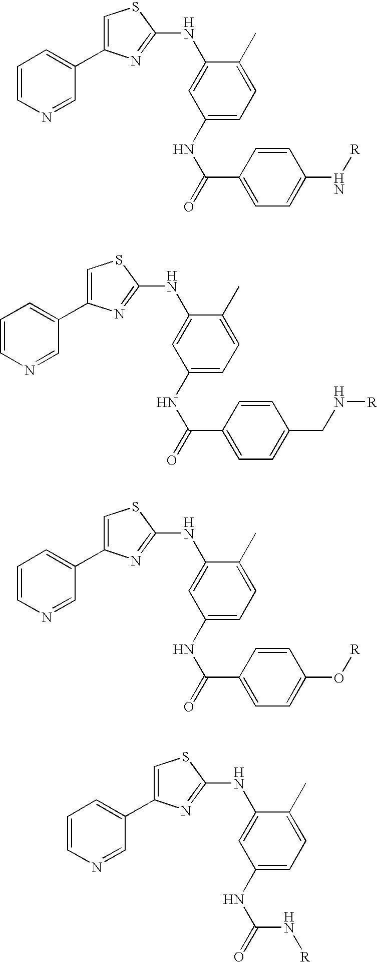

wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with a heteroatom, notably a halogen selected from I, Cl, Br and F and/or bearing a pendant basic nitrogen functionality; or a a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;

a —SO2-R group wherein R is an alkyl, cycloalkyl, aryl or heteroaryl optionally substituted with an heteroatom, notably a halogen selected from I, Cl, Br and F and/or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Among the particular compounds in which R1 has the meaning as depicted in a) and b) above, the invention is directed to N-Aminoalkyl-N′-thiazol-2-yl-benzene-1,3-diamine compounds of the following formula IV bis:

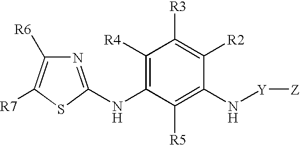

wherein Y is a linear or branched alkyl group containing from 1 to 10 carbon atoms; wherein Z represents an aryl or heteroaryl group, optionally substituted at one or more ring position with any permutation of the following groups:
a halogen such as F, Cl, Br, I;
a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an O—R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaRb, where Ra and Rb represents a hydrogen, or a linear or branched alkyl group containing from 1 to 10 carbon atoms atoms optionally substituted with at least one heteroatom (for example a halogen) and/or bearing a pendant basic nitrogen functionality or a cycle; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;

R² is hydrogen, halogen or a linear or branched alkyl group containing from 1 to 10 carbon atoms, trifluoromethyl or alkoxy;

R³ is hydrogen, halogen or a linear or branched alkyl group containing from 1 to 10 carbon atoms, trifluoromethyl or alkoxy;

R⁴ is hydrogen, halogen or a linear or branched alkyl group containing from 1 to 10 carbon atoms, trifluoromethyl or alkoxy;

R⁵ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality;

and R⁷ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, an halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

It will be understood that a C1-C10 alkyl encompasses a methyl, ethyl, propyl, and a C2 to C4 alkyl or a C2 to C10 alkyl.

An example of preferred compounds of the above formula is depicted below:

4-{[4-Methyl-3-(4-pyridin-3-yl-thiazol-2-ylamino)-phenylamino]-methyl}-benzoic acid methyl ester

Among the compounds of formula III or IV, the invention is particularly embodied by the compounds of the following formula V:

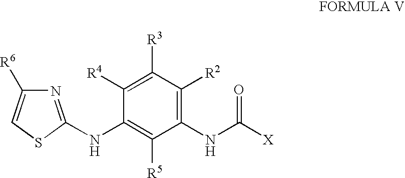

wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,

R² is hydrogen, halogen or a linear or branched alkyl group containing from 1 to 10 carbon atoms, trifluoromethyl or alkoxy;

R³ is hydrogen, halogen or a linear or branched alkyl group containing from 1 to 10 carbon atoms, trifluoromethyl or alkoxy;

R⁴ is hydrogen, halogen or a linear or branched alkyl group containing from 1 to 10 carbon atoms, trifluoromethyl or alkoxy;

R⁵ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula II is connected to position 4 of the thiazole ring, may instead occupy position 5 of the thiazole ring.

Among the preferred compounds corresponding formula III, IV or V, the invention is directed to compounds in which R1 or X is a substituted alkyl, aryl or heteroaryl group bearing a pendant basic nitrogen functionality represented for example by the structures a to f and g to m shown below, wherein the wavy line corresponds to the point of attachment to core structure of formula III, IV or V:

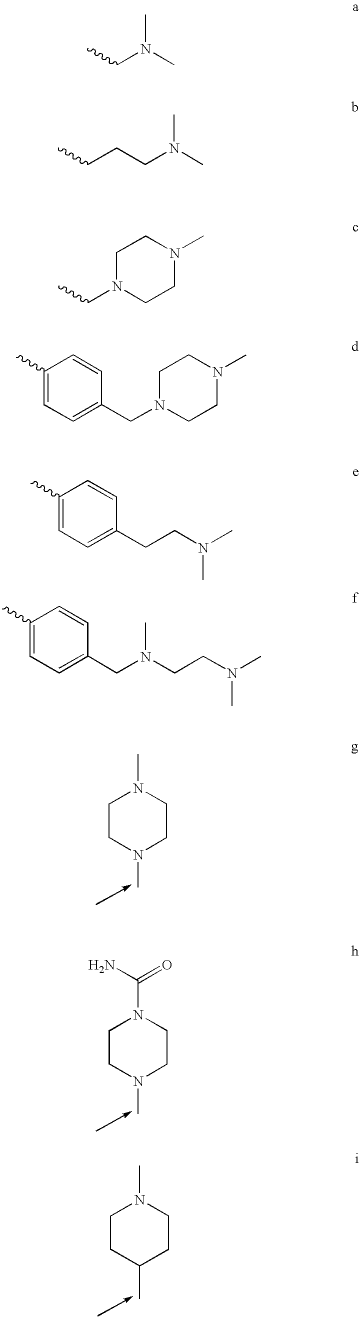

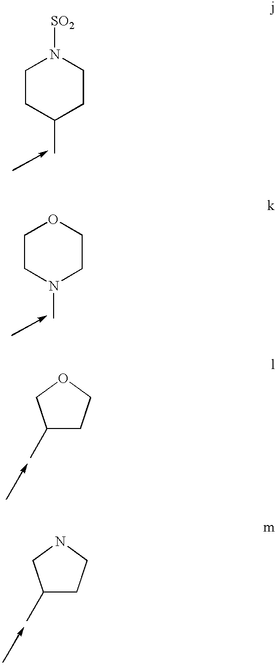

Among group a to f, is preferentially group d. Also, for g to m, the arrow may include a point of attachment to the core structure via a phenyl group.

Furthermore, among the preferred compounds of formula III, IV or V, the invention concerns the compounds in which R² and R³ are hydrogen. Preferentially, R⁴ is a methyl group and R⁵ is H. In addition, R⁶ is preferentially a 3-pyridyl group (cf. structure g below), or a 4-pyridyl group (cf. structure h below) or a benzonitrile group. The wavy line in structure g and h correspond to the point of attachment to the core structure of formula III, IV or V.

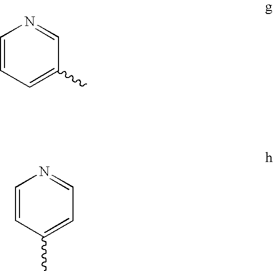

Alternatively, among the preferred compounds of formula III, IV or V, the invention concerns the compounds in which R6 or R7 is preferentially a cyanophenyl group as shown below, wherein the wavy line in structure p and q correspond to the point of attachment to the core structure of formula III, IV or V:

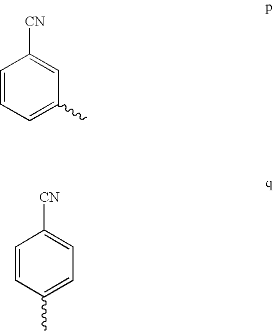

In one particular embodiment, R1 in formula III and IV, X in formula V and Z in formula IVbis can be:

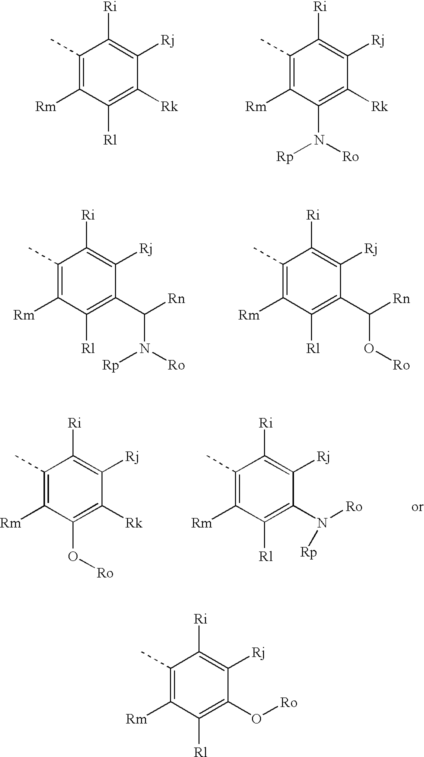

wherein Ri, Rj, Rk, Rl, Rm, Ro, and Rp are independently chosen from
H, an halogen such as Cl, F, Br, I; a trifluoromethyl group, a CN group, SO2, OH, or a group selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a NRR′, NRCOR, NRCONR′R″, NROSO₂R′, SO2-R, COOR, CONRR′, NHCOOR, CO—R, CO—NRR′, OR or OSO₂R group where R and R′ are independently chosen from H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality.

For example, one of Ri, Rj, Rk, Rl, Rm, Ro or Rp is selected from group a, b, c, g, h, i, j, k, l, m as defined above such as Rk is one of a, b, c, g, h, i, j, k, l, m and Ri, Rj, Rl, Rm is H.

Thus, the invention contemplates:
1—A compound of formula V as depicted above, wherein X is group d and R⁶ is a 3-pyridyl group.
2—A compound of formula V as depicted above, wherein X is group d and R⁴ is a methyl group.
3—A compound of formula III or IV as depicted above, wherein R¹ is group d and R² and/or R³ and/or R⁵ is H.
4—A compound of formula III or IV as depicted above, wherein R⁶ is a 3-pyridyl group and R⁴ is a methyl group.
5—A compound of formula III or IV as depicted above, wherein R² and/or R³ and/or R⁵ is H and R⁴ is a methyl group.
6—A compound of formula III or IV as depicted above wherein R² and/or R³ and/or R⁵ is H, R⁴ is a methyl group and R⁶ is a 3-pyridyl group.

Among the compounds of formula IV, the invention is particularly embodied by the compounds wherein R2, R3, R5 are hydrogen, corresponding to the following formula

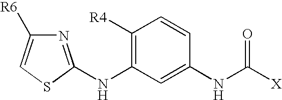

wherein X is R or NRR′ and wherein R and R′ are independently chosen from H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with an heteroatom, notably a halogen selected from I, Cl, Br and F or bearing a pendant basic nitrogen functionality; or a a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;

a —SO2-R group wherein R is an alkyl, cycloalkyl, aryl or heteroaryl optionally substituted with a heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula III is connected to position 4 of the thiazole ring, may instead occupy position 5 of the thiazole ring.

### EXAMPLES

2-(2-methyl-5-amino)phenyl-4-(3-pyridyl)-thiazole

4-(4-Methyl-piperazin-1-ylmethyl)-N-[3-(4-pyridin-3-yl-thiazol-2-ylamino)-phenyl]-benzamide

N-[4-Methyl-3-(4-phenyl-thiazol-2-ylamino)-phenyl]-4-(4-methyl-piperazin-1-ylmethyl)-benzamide

N-[3-([2,4′]Bithiazolyl-2′-ylamino)-4-methyl-phenyl]-4-(4-methyl-piperazin-1-ylmethyl)-benzamide

4-(4-Methyl-piperazin-1-ylmethyl)-N-[4-methyl-3-(4-pyrazin-2-yl-thiazol-2-ylamino)-phenyl]-benzamide

2-[5-(3-Iodo-benzoylamino)-2-methyl-phenylamino]-thiazole-4-carboxylic acid ethyl ester

2-{2-Methyl-5-[4-(4-methyl-piperazin-1-ylmethyl)-benzoylamino]-phenylamino}-thiazole-4-carboxylic acid ethyl ester

2-(2-chloro-5-amino)phenyl-4-(3-pyridyl)-thiazole

3-Bromo-N-{3-[4-(4-chloro-phenyl)-5-methyl-thiazol-2-ylamino]-4-methyl-phenyl}-benzamide

{3-[4-(4-Chloro-phenyl)-5-methyl-thiazol-2-ylamino]-4-methyl-phenyl}-carbamic acid isobutyl ester

2-[5-(3-Bromo-benzoylamino)-2-methyl-phenylamino]-5-(4-chloro-phenyl)-thiazole-4-carboxylic acid ethyl ester

2-[5-(3-Bromo-benzoylamino)-2-methyl-phenylamino]-5-(4-chloro-phenyl)-thiazole-4-carboxylic acid (2-dimethylamino-ethyl)-amide

N-{3-[4-(4-Methoxy-phenyl)-thiazol-2-ylamino]-4-methyl-phenyl}-4-(4-methyl-piperazin-1-ylmethyl)-benzamide

4-(4-Methyl-piperazin-1-ylmethyl)-N-{4-methyl-3-[4-(3-trifluoromethyl-phenyl)-thiazol-2-ylamino]-phenyl}-benzamide

N-{4-Methyl-3-[4-(3-nitro-phenyl)-thiazol-2-ylamino]-phenyl}-4-(4-methyl-piperazin-1-ylmethyl)-benzamide

N-{3-[4-(2,5-Dimethyl-phenyl)-thiazol-2-ylamino]-4-methyl-phenyl}-4-(4-methyl-piperazin-1-ylmethyl)-benzamide

N-{3-[4-(4-Chloro-phenyl)-thiazol-2-ylamino]-4-methyl-phenyl}-4-(4-methyl-piperazin-1-ylmethyl)-benzamide

N-{3-[4-(3-Methoxy-phenyl)-thiazol-2-ylamino]-4-methyl-phenyl}-4-(4-methyl-piperazin-1-ylmethyl)-benzamide

N-[4-Methyl-3-(4-pyridin-3-yl-thiazol-2-ylamino)-phenyl]-isonicotinamide

2,6-Dichloro-N-[4-methyl-3-(4-pyridin-3-yl-thiazol-2-ylamino)-phenyl]-isonicotinamide

3-Phenyl-propynoic acid [4-methyl-3-(4-pyridin-3-yl-thiazol-2-ylamino)-phenyl]-amide

Cyclohexanecarboxylic acid [4-methyl-3-(4-pyridin-3-yl-thiazol-2-ylmethyl)-phenyl]-amide

5-[4-Methyl-3-(4-pyridin-3-yl-thiazol-2-ylamino)-phenylcarbamoyl]-pentanoic acid ethyl ester

1-Methyl-cyclohexanecarboxylic acid [4-methyl-3-(4-pyridin-3-yl-thiazol-2-ylmethyl)-phenyl]-amide

4-tert-Butyl-cyclohexanecarboxylic acid [4-methyl-3-(4-pyridin-3-yl-thiazol-2-ylamino)-phenyl]-amide

N-[4-Methyl-3-(4-pyridin-3-yl-thiazol-2-ylamino)-phenyl]-4-morpholin-4-yl-butyramide

Among the compounds of formula IV, the invention is particularly embodied by the compounds wherein X is a urea group, a —CO—NRR′ group, corresponding to the [3-(thiazol-2-ylamino)-phenyl]-urea family and the following formula:

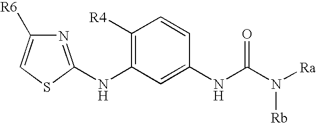

wherein Ra, Rb are independently chosen from Y-Z as defined above or

H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F or bearing a pendant basic nitrogen functionality;

a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, or bearing a pendant basic nitrogen functionality.

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### Example 1

1-(4-Methoxy-phenyl)-3-[4-methyl-3-(4-pyridin-3-yl-thiazol-2-ylamino)-phenyl]-urea

1-(4-Bromo-phenyl)-3-[4-methyl-3-(4-pyridin-3-yl-thiazol-2-ylamino)-phenyl]-urea

1-[4-Methyl-3-(4-pyridin-3-yl-thiazol-2-ylamino)-phenyl]-3-(4-trifluoromethyl-phenyl)-urea

1-(4-Fluoro-phenyl)-3-[4-methyl-3-(4-pyridin-3-yl-thiazol-2-ylamino)-phenyl]-urea

1-[4-Methyl-3-(4-pyridin-3-yl-thiazol-2-ylamino)-phenyl]-3-(3,4,5-trimethoxy-phenyl)-urea
4-{3-[4-Methyl-3-(4-pyridin-3-yl-thiazol-2-ylamino)-phenyl]-ureido}-benzoic acid ethyl ester

1-[4-Methyl-3-(4-pyridin-3-yl-thiazol-2-ylamino)-phenyl]-3-thiophen-2-yl-urea

1-Cyclohexyl-1-(N-Cyclohexyl-formamide)-3-[4-methyl-3-(4-pyridin-3-yl-thiazol-2-ylamino)-phenyl]-urea

1-(2,4-Dimethoxy-phenyl)-3-[4-methyl-3-(4-pyridin-3-yl-thiazol-2-ylamino)-phenyl]-urea

1-(2-Iodo-phenyl)-1-(N-(2-Iodo-phenyl)-formamide)-3-[4-methyl-3-(4-pyridin-3-yl-thiazol-2-ylamino)-phenyl]-urea

1-(3,5-Dimethyl-isoxazol-4-yl)-3-[4-methyl-3-(4-pyridin-3-yl-thiazol-2-ylamino)-phenyl]urea

1-(2-Iodo-phenyl)-3-[4-methyl-3-(4-pyridin-3-yl-thiazol-2-ylamino)-phenyl]-urea

1-(4-Difluoromethoxy-phenyl)-3-[4-methyl-3-(4-pyridin-3-yl-thiazol-2-ylamino)-phenyl]-urea

1-(4-Dimethylamino-phenyl)-3-[4-methyl-3-(4-pyridin-3-yl-thiazol-2-ylamino)-phenyl]-urea

1-(2-Fluoro-phenyl)-3-[4-methyl-3-(4-pyridin-3-yl-thiazol-2-ylamino)-phenyl]-urea

1-(2-Chloro-phenyl)-3-[4-methyl-3-(4-pyridin-3-yl-thiazol-2-ylamino)-phenyl]-urea

1-(3-Fluoro-phenyl)-3-[4-methyl-3-(4-pyridin-3-yl-thiazol-2-ylamino)-phenyl]-urea

1-[4-Methyl-3-(4-pyridin-3-yl-thiazol-2-ylamino)-phenyl]-3-p-tolyl-urea

3-Bromo-N-[4-methyl-3-(4-pyridin-3-yl-thiazol-2-ylamino)-phenyl]-benzamide

3-Iodo-N-[4-methyl-3-(4-pyridin-3-yl-thiazol-2-ylamino)-phenyl]-benzamide

4-Hydroxymnethyl-N-[4-methyl-3-(4-pyridin-3-yl-thiazol-2-ylamino)-phenyl]-benzamide

4-Amino-N-[4-methyl-3-(4-pyridin-3-yl-thiazol-2-ylamino)-phenyl]-benzamide

2-Iodo-N-[4-methyl-3-(4-pyridin-3-yl-thiazol-2-ylamino)-phenyl]-benzamide

4-Iodo-N-[4-methyl-3-(4-pyridin-3-yl-thiazol-2-ylamino)-phenyl]-benzamide

4-(3-{4-[4-Methyl-3-(4-pyridin-3-yl-thiazol-2-ylamino)-phenylcarbamoyl]-phenyl}-ureido)-benzoic acid ethyl ester

N-[4-Methyl-3-(4-pyridin-3-yl-thiazol-2-ylamino)-phenyl]-4-[3-(4-trifluoromethyl-phenyl)-ureido]-benzamide

4-[3-(4-Bromo-phenyl)-ureido]-N-[4-methyl-3-(4-pyridin-3-yl-thiazol-2-ylamino)-phenyl]-benzamide

4-Hydroxy-N-[4-methyl-3-(4-pyridin-3-yl-thiazol-2-ylamino)-phenyl]-benzamide

N-[4-Methyl-3-(4-pyridin-3-yl-thiazol-2-ylamino)-phenyl]-4-(3-thiophen-2-yl-ureido)-benzamide

4-[3-(3,5-Dimethyl-isoxazol-4-yl)-ureido]-N-[4-methyl-3-(4-pyridin-3-yl-thiazol-2-ylamino)-phenyl]-benzamide

4-[3-(4-Methoxy-phenyl)-ureido]-N-[4-methyl-3-(4-pyridin-3-yl-thiazol-2-ylamino)-phenyl]-benzamide

4-[3-(4-Difluoromethoxy-phenyl)-ureido]-N-[4-methyl-3-(4-pyridin-3-yl-thiazol-2-ylamino)-phenyl]-benzamide

Thiophene-2-sulfonic acid 4-[4-methyl-3-(4-pyridin-3-yl-thiazol-2-ylamino)-phenylcarbamoyl]-phenyl ester

4-Iodo-benzenesulfonic acid 4-[4-methyl-3-(4-pyridin-3-yl-thiazol-2-ylamino)-phenylcarbamoyl]-phenyl ester

N-[4-Methyl-3-(4-pyridin-3-yl-thiazol-2-ylamino)-phenyl]-4-(thiophene-2-sulfonylamino)-benzamide

3-Fluoro-N-[4-methyl-3-(4-pyridin-3-yl-thiazol-2-ylamino)-phenyl]-benzamide

N-[4-Methyl-3-(4-pyridin-3-yl-thiazol-2-ylamino)-phenyl]-4-pyridin-4-yl-benzamide

4-Dimethylamino-N-[4-methyl-3-(4-pyridin-3-yl-thiazol-2-ylamino)-phenyl]-benzamide

2-Fluoro-5-methyl-N-[4-methyl-3-(4-pyridin-3-yl-thiazol-2-ylamino)-phenyl]-benzamide

4-tert-Butyl-N-[4-methyl-3-(4-pyridin-3-yl-thiazol-2-ylamino)-phenyl]-benzamide

4-Isopropoxy-N-[4-methyl-3-(4-pyridin-3-yl-thiazol-2-ylmethyl)-phenyl]-benzamide

Benzo[1,3]dioxole-5-carboxylic acid [4-methyl-3-(4-pyridin-3-yl-thiazol-2-ylmethyl)-phenyl]-amide

N-[4-Methyl-3-(4-pyridin-3-yl-thiazol-2-ylamino)-phenyl]-3-(2-morpholin-4-yl-ethoxy)-benzamide

N-[4-Methyl-3-(4-pyridin-3-yl-thiazol-2-ylmethyl)-phenyl]-4-pyridin-4-yl-benzamide

3-Cyano-N-[4-methyl-3-(4-pyridin-3-yl-thiazol-2-ylamino)-phenyl]-benzamide

2-Fluoro-N-[4-methyl-3-(4-pyridin-3-yl-thiazol-2-ylamino)-phenyl]-3-trifluoromethyl-benzamide

3-Fluoro-benzenesulfonic acid 4-[4-methyl-3-(4-pyridin-3-yl-thiazol-2-ylamino)-phenylcarbamoyl]-phenyl ester

4-Aminomethyl-N-[4-methyl-3-(4-pyridin-3-yl-thiazol-2-ylamino)-phenyl]-benzamide

2-Fluoro-benzenesulfonic acid 4-[4-methyl-3-(4-pyridin-3-yl-thiazol-2-ylamino)-phenylcarbamoyl]-phenyl ester

3-Methoxy-N-[4-methyl-3-(4-pyridin-3-yl-thiazol-2-ylmethyl)-phenyl]-benzamide

4-(4-Methyl-piperazin-1-yl)-N-[4-methyl-3-(4-pyridin-3-yl-thiazol-2-ylmethyl)-phenyl]-benzamide

3-Methyl-N-[4-methyl-3-(4-pyridin-3-yl-thiazol-2-ylamino)-phenyl]-benzamide

Biphenyl-3-carboxylic acid [4-methyl-3-(4-pyridin-3-yl-thiazol-2-ylamino)-phenyl]-amide

N-[4-Methyl-3-(4-pyridin-3-yl-thiazol-2-ylamino)-phenyl]-3-trifluoromethyl-benzamide

N-[4-Methyl-3-(4-pyridin-3-yl-thiazol-2-ylamino)-phenyl]-4-pyrrolidin-1-ylmethyl-benzamide

4-[3-(2,4-Dimethoxy-phenyl)-ureido]-N-[4-methyl-3-(4-pyridin-3-yl-thiazol-2-ylamino)-phenyl]-benzamide

4-[3-(2-Iodo-phenyl)-ureido]-N-[4-methyl-3-(4-pyridin-3-yl-thiazol-2-ylamino)-phenyl]-benzamide

4-[3-(4-Fluoro-phenyl)-ureido]-N-[4-methyl-3-(4-pyridin-3-yl-thiazol-2-ylamino)-phenyl]-benzamide

3-Bromo-4-methyl-N-[4-methyl-3-(4-pyridin-3-yl-thiazol-2-ylamino)-phenyl]-benzamide

4-Fluoro-N-[4-methyl-3-(4-pyridin-3-yl-thiazol-2-ylamino)-phenyl]-benzamide

4-Cyano-N-[4-methyl-3-(4-pyridin-3-yl-thiazol-2-ylamino)-phenyl]-benzamide

4-Fluoro-N-[4-methyl-3-(4-pyridin-3-yl-thiazol-2-ylamino)-phenyl]-benzamide

### Example 2

4-(4-methyl-piperazin-1-ylmethyl)-N-[4-methyl-3-(4-pyridin-3-yl-thiazol-2-ylamino)-phenyl]-benzamide

3,5-Dibromo-4-(4-methyl-piperazin-1-ylmethyl)-N-[4-methyl-3-(4-pyridin-3-yl-thiazol-2-ylamino)-phenyl]-benzamide

4-Diethylaminomethyl-N-[4-methyl-3-(4-pyridin-3-yl-thiazol-2-ylamino)-phenyl]-benzamide

N-[4-Methyl-3-(4-pyridin-3-yl-thiazol-2-ylamino)-phenyl]-4-morpholin-4-ylmethyl-benzamide

4-Dipropylaminomethyl-N-[4-methyl-3-(4-pyridin-3-yl-thiazol-2-ylamino)-phenyl]-benzamide

N-[4-Methyl-3-(4-pyridin-3-yl-thiazol-2-ylamino)-phenyl]-4-piperidin-1-ylmethyl-benzamide

4-[(Diisopropylamino)-methyl]-N-[4-methyl-3-(4-pyridin-3-yl-thiazol-2-ylamino)-phenyl]-benzamide

{4-[4-Methyl-3-(4-pyridin-3-yl-thiazol-2-ylamino)-phenylcarbamoyl]-benzyl}-carbamic acid tert-butyl ester

3-Fluoro-4-(4-methyl-piperazin-1-ylmethyl)-N-[4-methyl-3-(4-pyridin-3-yl-thiazol-2-ylamino)-phenyl]-benzamide

4-(4-Methyl-piperazin-1-ylmethyl)-N-[4-methyl-3-(4-pyridin-3-yl-thiazol-2-ylmethyl)-phenyl[-3-trifluoromethyl-benzamide

2,3,5,6-Tetrafluoro-4-(4-methyl-piperazin-1-ylmethyl)-N-[4-methyl-3-(4-pyridin-3-yl-thiazol-2-ylamino)-phenyl]-benzamide

N-{3-[4-(4-Fluoro-phenyl)-thiazol-2-ylamino]-4-methyl-phenyl}-4-(4-methyl-piperazin-1-ylmethyl)-benzamide

3-Bromo-4-(4-methyl-piperazin-1-ylmethyl)-N-[4-methyl-3-(4-pyridin-3-yl-thiazol-2-ylamino)-phenyl]-benzamide

3-Chloro-4-(4-methyl-piperazin-1-ylmethyl)-N-[4-methyl-3-(4-pyridin-3-yl-thiazol-2-ylamino)-phenyl]-benzamide

4-(4-Methyl-piperazin-1-ylmethyl)-N-[4-methyl-3-(4-pyridin-4-yl-thiazol-2-ylamino)-phenyl]-benzamide

N-{3-[4-(4-Cyano-phenyl)-thiazol-2-ylamino]-4-methyl-phenyl}-4-(4-methyl-piperazin-1-ylmethyl)-benzamide

4-[1-(4-Methyl-piperazin-1-yl)-ethyl]-N-[4-methyl-3-(4-pyridin-3-yl-thiazol-2-ylmethyl)-phenyl]-benzamide

4-(1-Methoxy-ethyl)-N-[4-methyl-3-(4-pyridin-3-yl-thiazol-2-ylmethyl)-phenyl]-benzamide

N-{4-Methyl-3-[4-(5-methyl-pyridin-3-yl)-thiazol-2-ylamino]-phenyl}-4-(4-methyl-piperazin-1-ylmethyl)-benzamide

3-Iodo-4-(4-methyl-piperazin-1-ylmethyl)-N-[4-methyl-3-(4-pyridin-3-yl-thiazol-2-ylmethyl)-phenyl]-benzamide

N-[4-Methyl-3-(4-pyridin-3-yl-thiazol-2-ylamino)-phenyl]-4-[3-(4-trifluoromethyl-phenyl)-ureidomethyl]-benzamide

3,5-Dibromo-N-[4-methyl-3-(4-pyridin-3-yl-thiazol-2-ylamino)-phenyl]-4-[(3-morpholin-4-yl-propylamino)-methyl]-benzamide

3,5-Dibromo-N-[4-methyl-3-(4-pyridin-3-yl-thiazol-2-ylamino)-phenyl]-4-piperidin-1-ylmethyl-benzamide

4-(4-Methyl-piperazin-1-ylmethyl)-N-[4-methyl-3-(4-pyridin-2-yl-thiazol-2-ylamino)-phenyl]-benzamide

N-{3-[4-(3-Fluoro-phenyl)-thiazol-2-ylamino]-4-methyl-phenyl}-4-(4-methyl-piperazin-1-ylmethyl)-benzamide

N-{3-[4-(2-Fluoro-phenyl)-thiazol-2-ylamino]-4-methyl-phenyl}-4-(4-methyl-piperazin-1-ylmethyl)-benzamides

### Example 3

3-Dimethylamino-N-[4-methyl-3-(4-pyridin-3-yl-thiazol-2-ylamino)-phenyl]-benzamide

3-(4-Methyl-piperazin-1-yl)-N-[4-methyl-3-(4-pyridin-3-yl-thiazol-2-ylamino)-phenyl]-benzamide

N-[4-Methyl-3-(4-pyridin-3-yl-thiazol-2-ylamino)-phenyl]-3-morpholin-4-yl-benzamide

Among the compounds of formula IV, the invention is particularly embodied by the compounds wherein X is a —OR group, corresponding to the family [3-(Thiazol-2-ylamino)-phenyl]-carbamate and the following formula IV-6

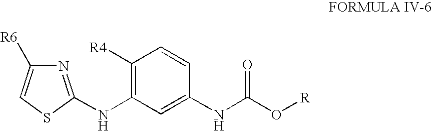

wherein R is independently chosen from an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with an heteroatom, notably a halogen selected from I, Cl, Br and F and/or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;

R4 and R6 are as defined above.

In still another preferred embodiment, the invention contemplated the method mentioned above, wherein said c-kit inhibitor is selected from 2-aminoaryloxazoles of formula X:

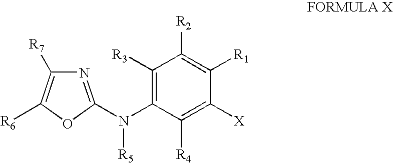

wherein substituents R1-R7 and X are defined as follows:

R1, R2, R3 and R4 each independently are selected from hydrogen, halogen (selected from F, Cl, Br or ), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R5 is one of the following:

(i) hydrogen, or

(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or

(iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or D), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R6 and R7 each independently are selected from:

i) hydrogen, a halogen (selected from F, Cl, Br or I), or

ii) an alkyl¹ group defined as a linear, branched or cycloalkyl group containing from 1 to 10 carbon atoms and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality ; as well as a cycloalkyl or aryl or heteroaryl group optionally substituted by a a pendant basic nitrogen functionality, or

(iii) an aryl¹ group defined as phenyl or a substituted variant thereof bearing any combination, at any one ring position, of one or more substituents such as
halogen(selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, or

(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I;
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, or

(v) an O-aryl¹, or NH-aryl¹, or O-heteroaryl¹ or NH-heteroaryl¹ group

(vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or

(vi) NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl.

X is:

—NR9R10, wherein R9 and or R10 are hydrogen or:

i) an alkyl¹ group, CF3 or

ii) an aryl¹, heteroaryl¹ or cycloalkyl group optionally substituted by a a pendant basic nitrogen functionality, or

iii) a CO—R, COO—R, CON—RR′ or SO2-R, where R and R′ are a hydrogen, alkyl¹, aryl¹ or heteroaryl¹, optionally substituted by a a pendant basic nitrogen functionality; or:

—CO—NR9R10, wherein R9 and/or R10 are hydrogen or:

i) an alkyl¹ group, CF3 or

ii) an aryl¹, heteroaryl¹ or cycloalkyl group optionally substituted by a a pendant basic nitrogen functionality.

Such compound may be selected from N-Aminoalkyl-N′-oxazol-2-yl-benzene-1,3-diamines of the following formula:

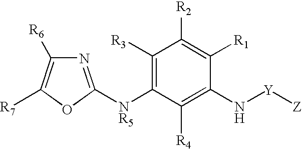

wherein R5=H, Y is a linear or branched alkyl group containing from 1 to 10 carbon atoms and Z represents an aryl or a heteroaryl group, optionally substituted by a pendant basic nitrogen functionality.

For example, it is the 4-{[4-Methyl-3-(4-pyridin-3-yl-oxazol-2-ylamino)-phenylamino]-methyl}-benzoic acid methyl ester.

The above 2-aminoaryloxazoles compounds may have the formula XI:

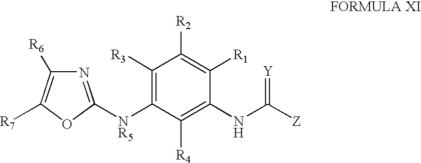

Wherein R5 is H, Y is selected from O, S and Z corresponds to H, alkyl, or NRR′, wherein R and R′ are independently chosen from H or alkyl¹ or aryl¹ or heteroaryl¹, optionally substituted by a pendant basic nitrogen functionality, for example:

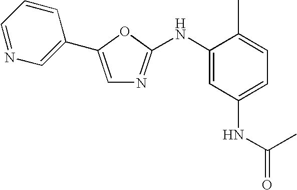

or a compound of formula XI-1:

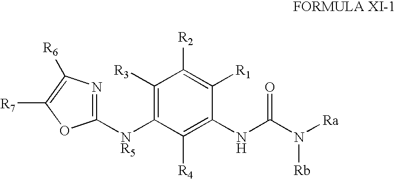

wherein Ra, Rb are independently chosen from H or alkyl¹ or aryl¹ or heteroaryl¹, optionally substituted by a pendant basic nitrogen functionality, for example:

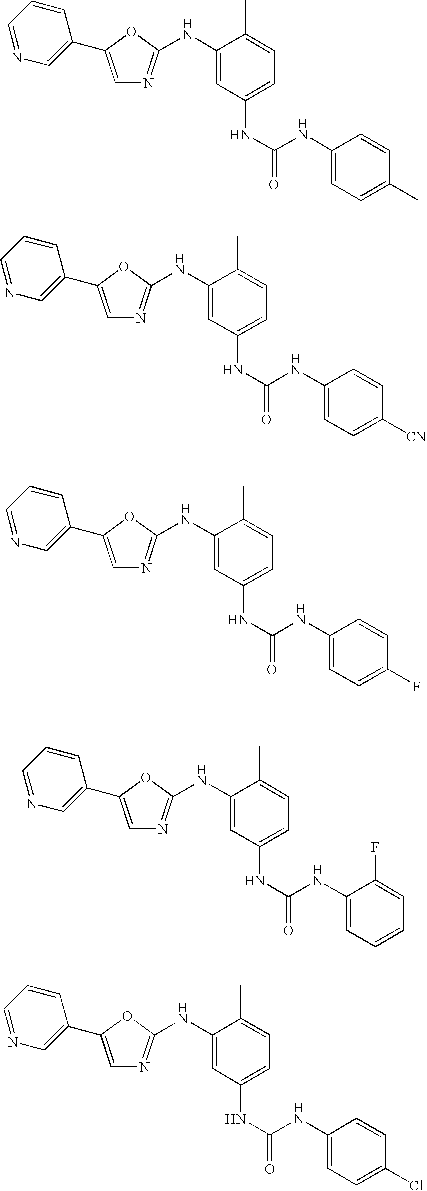

or a compound of formula XI-2:

FORMULA XI-2

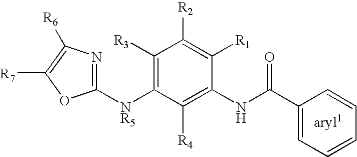

wherein R5=H, Z is an aryl¹ group, aryl¹ being selected from:

a phenyl or a substituted variant thereof bearing any combination, at any one ring position, of one or more substituents such as
halogen(selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;

NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, for example

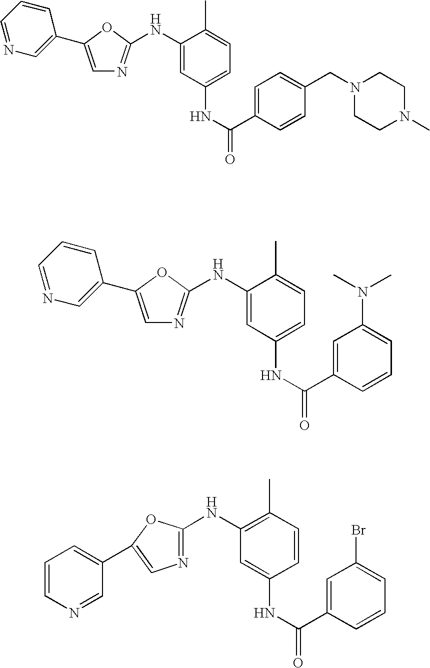

or a compound of formula XI-3:

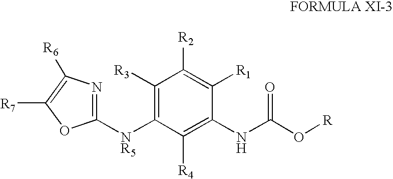

wherein R5=H and R is independently alkyl¹, aryl¹ or heteroaryl¹ as defined above.

Examples of Compounds of Formula X:

4-[4-Methyl-3-(4-pyridin-3-yl-oxazol-2-ylamino)-phenylamino]-methyl}-benzoic acid methyl ester

4-Methyl-N1-(5-pyridin-3-yl-oxazol-2-yl)-N3-(5-pyridin-4-yl-oxazol-2-yl)-benzene-1,3-diamine m.p.

4-Methyl-N1-(5-phenyl-oxazol-2-yl)-N3-(5-pyridin-4-yl-oxazol-2-yl)-benzene-1,3-diamine

4-Methyl-N1-(5-phenyl-[1,3,4]oxadiazol-2-yl)-N3-(5-pyridin-4-yl-oxazol-2-yl)-benzene-1,3-diamine

N1-Benzooxazol-2-yl-4-methyl-N3-(5-pyridin-4-yl-oxazol-2-yl)-benzene-1,3-diamine

N-[4-Methyl-3-(5-pyridin-4-yl-oxazol-2-ylamino)-phenyl]-C-phenyl-methanesulfon-amide

N-[4-Methyl-3-(5-pyridin-3-yl-oxazol-2-ylamino)-phenyl]-acetamide

2-Cyano-N-[4-methyl-3-(5-pyridin-4-yl-oxazol-2-ylamino)-phenyl]-acetamide

2-Ethoxy-N-[4-methyl-3-(5-pyridin-3-yl-oxazol-2-ylamino)-phenyl]-acetamide

3-Methoxy-N-[4-methyl-3-(5-pyridin-3-yl-oxazol-2-ylamino)-phenyl]-propionamide

1-(4-Cyano-phenyl)-3-[4-methyl-3-(5-pyridin-3-yl-oxazol-2-ylamino)-phenyl]-urea

1-(4-Fluoro-phenyl)-3-[4-methyl-3-(5-pyridin-3-yl-oxazol-2-ylamino)-phenyl]-urea

1-(2-Fluoro-phenyl)-3-[4-methyl-3-(5-pyridin-3-yl-oxazol-2-ylamino)-phenyl]-urea

1-[4-Methyl-3-(5-pyridin-3-yl-oxazol-2-ylamino)-phenyl]-3-(4-trifluoromethyl-phenyl)-urea

1-(4-Chloro-phenyl)-3-[4-methyl-3-(5-pyridin-3-yl-oxazol-2-ylamino)-phenyl]-urea

1-[4-Methyl-3-(5-phenyl-oxazol-2-ylamino)-phenyl]-3-(3-trifluoromethyl-phenyl)-urea

1-(4-Cyano-phenyl)-3-[4-methyl-3-(5-pyridin-3-yl-oxazol-2-ylamino)-phenyl]-thiourea

1-(4-Cyano-phenyl)-3-[4-methyl-3-(5-pyridin-4-yl-oxazol-2-ylamino)-phenyl]-thiourea

(2-{2-Methyl-5-[3-(4-trifluoromethyl-phenyl)-ureido]-phenylamino}-oxazol-5-yl)-acetic acid ethyl ester

1-Benzyl-3-[4-methyl-3-(-pyridin-4-yl-oxazol-2-ylamino)-phenyl]-thiourea

4-(4-Methyl-piperazin-1-ylmethyl)-N-[4-methyl-3-(5-pyridin-3-yl-oxazol-2-ylamino)-phenyl]-benzamide

3-Dimethylamino-N-[4-methyl-3-(5-pyridin-3-yl-oxazol-2-ylamino)-phenyl]-benzamide

3-Bromo-N-[4-methyl-3-(5-pyridin-3-yl-oxazol-2-ylamino)-phenyl]-benzamide

N-[4-Methoxy-3-(5-pyridin-3-yl-oxazol-2-ylamino)-phenyl]-3-trifluoromethyl-benzamide

4-(3-Dimethylamino-propylamino)-N-[4-methyl-3-(5-pyridin-3-yl-oxazol-2-ylamino)-phenyl]-3-trifluoromethyl-benzamide

N-[4-Fluoro-3-(5-pyridin-3-yl-oxazol-2-ylamino)-phenyl]-3-trifluoromethyl-benzamide

1H-Indole-6-carboxylic acid [4-methyl-3-(5-pyridin-4-yl-oxazol-2-ylamino)-phenyl]-amide

3-Isopropoxy-N-[4-methyl-3-(5-pyridin-4-yl-oxazol-2-ylamino)-phenyl]-benzamide

N-[4-Methyl-3-(5-pyridin-2-yl-oxazol-2-ylamino)-phenyl]-3-trifluoromethyl-benzamide

3,5-Dimethoxy-N-[4-methyl-3-(5-pyridin-4-yl-oxazol-2-ylamino)-phenyl]-benzamide

N-[3-(5-Pyridin-3-yl-oxazol-2-ylamino)-phenyl]-3-trifluoromethyl-benzamide

N-[4-Methyl-3-(5-phenyl-oxazol-2-ylamino)-phenyl]-3-trifluoromethyl-benzamide

3-Fluoro-4-(4-methyl-piperazin-1-ylmethyl)-N-[4-methyl-3-(5-pyridin-3-yl-oxazol-2-ylamino)-phenyl]-benzamide

N-[4-Chloro-3-(5-pyridin-3-yl-oxazol-2-ylamino)-phenyl]-3-trifluoromethyl-benzamide

N-[4-Methyl-3-(5-pyridin-3-yl-oxazol-2-ylamino)-phenyl]-terephthalamide

5-Methyl-isoxazole-4-carboxylic acid [4-methyl-3-(5-pyridin-4-yl-oxazol-2-ylamino)-phenyl]-amide

4-Cyano-N-[4-methyl-3-(5-pyridin-4-yl-oxazol-2-ylamino)-phenyl]-benzamide

N-[4-Methyl-3-(5-pyridin-3-yl-oxazol-2-ylamino)-phenyl]-isonicotinamide

N-[4-Methyl-3-(4-pyridin-3-yl-oxazol-2-ylamino)-phenyl]-3-trifluoromethyl-benzamide

[4-Methyl-3-(5-pyridin-3-yl-oxazol-2-ylamino)-phenyl]-carbamic acid isobutyl ester

(5-Isobutoxycarbonylamino-2-methyl-phenyl)-(5-pyridin-3-yl-oxazol-2-yl)-carbamic acid isobutyl ester

[4-Methyl-3-(5-pyridin-4-yl-oxazol-2-ylamino)-phenyl]-carbamic acid isobutyl ester

N-[4-Methyl-3-(5-pyridin-4-yl-oxazol-2-ylamino)-phenyl]-2-m-tolyl-acetamide

2-(4-Fluoro-phenyl)-N-[4-methoxy-3-(5-pyridin-4-yl-oxazol-2-ylamino)-phenyl]-acetamide

2-(2,4-Difluoro-phenyl)-N-[4-methyl-3-(5-phenyl-oxazol-2-ylamino)-phenyl]-acetamide

2-(3-Bromo-phenyl)-N-[4-methyl-3-(5-pyridin-2-yl-oxazol-2-ylamino)-phenyl]-acetamide

3-(4-Fluoro-phenyl)-N-[4-methyl-3-(5-pyridin-4-yl-oxazol-2-ylamino)-phenyl]-propionamide

N-{3-[5-(4-Cyano-phenyl)-oxazol-2-ylamino]-4-methyl-phenyl}-2-(2,4-difluoro-phenyl)-acetamide

4-Methyl-pentanoic acid [4-methyl-3-(5-pyridin-3-yl-oxazol-2-ylamino)-phenyl]-amide

N-[4-Methyl-3-(5-pyridin-3-yl-oxazol-2-ylamino)-phenyl]-2-piperazin-1-yl-acetamide

N-[4-Methyl-3-(5-pyridin-3-yl-oxazol-2-ylamino)-phenyl]-3-piperazin-1-yl-propionamide

2-(2,6-Dichloro-phenyl)-N-[4-methyl-3-(5-pyridin-4-yl-oxazol-2-ylamino)-phenyl]-acetamide

N-[4-Methyl-3-(5-pyridin-3-yl-oxazol-2-ylamino)-phenyl]-3-pyrrolidin-1-yl-propionamide

N-[4-Methoxy-3-(5-pyridin-4-yl-oxazol-2-ylamino)-phenyl]-2-(4-trifluoromethyl-phenyl)-acetamide

2-(4-Methoxy-phenyl)-N-[4-methyl-3-(5-pyridin-4-yl-oxazol-2-ylamino)-phenyl]-acetamide

N-(4-Cyano-phenyl)-4-methyl-3-(5-pyridin-3-yl-oxazol-2-ylamino)-benzamide

N-(3-Dimethylamino-phenyl)-4-methyl-3-(5-pyridin-4-yl-oxazol-2-ylamino)-benzamide

N-(2-Dimethylamino-ethyl)-4-methyl-3-(5-pyridin-3-yl-oxazol-2-ylamino)-benzamide

N-(3-Fluoro-4-methyl-phenyl)-4-methyl-3-(5-pyridin-4-yl-oxazol-2-ylamino)-benzamide

N-(3-Chloro-phenyl)-4-methyl-3-(5-pyridin-3-yl-oxazol-2-ylamino)-benzamide

N-Benzyl-4-methyl-3-(5-pyridin-4-yl-oxazol-2-ylamino)-benzamide

N-(4-Methoxy-benzyl)-4-methyl-3-(5-pyridin-4-yl-oxazol-2-ylamino)-benzamide

[4-Methyl-3-(5-pyridin-4-yl-oxazol-2-ylamino)-phenyl]-morpholin-4-yl-methanone

[4-Methyl-3-(5-pyridin-4-yl-oxazol-2-ylamino)-phenyl]-piperazin-1-yl-methanone

N-(4-Fluoro-phenyl)-2-[4-methyl-3-(5-pyridin-4-yl-oxazol-2-ylamino)-phenyl]-acetamide

Process for Manufacturing a Compound of Formula III Depicted Above

This entails the condensation of a substrate of general formula 10 with a thiourea of the type 11.

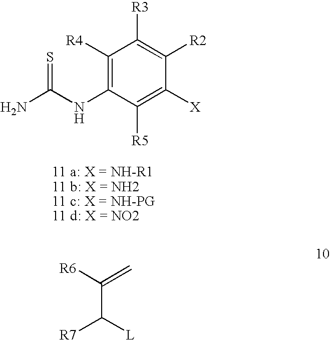

Substituent “L” in formula 10 is a nucleofugal leaving group in nucleophilic substitution reactions (for example, L can be selected from chloro, bromo, iodo, toluenesulfonyloxy, methanesulfonyloxy, trifluoromethanesulfonyloxy, etc., with L being preferentially a bromo group).

Group R1 in formula 11a corresponds to group R1 as described in formula III.

Group “PG” in formula 11c is a suitable protecting group of a type commonly utilized by the person skilled in the art.

The reaction of 10 with 1a-d leads to a thiozole-type product of formula 12a-d.

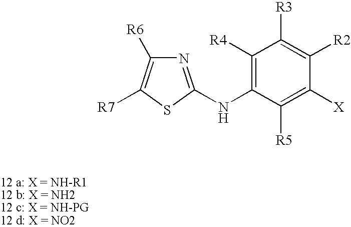

Formula 12a is the same as formula I. Therefore, R1 in 12a corresponds to R1 in formula III.

Formula 12b describes a precursor to compounds of formula III which lack substituent R1. Therefore, in a second phase of the synthesis, substituent R1 is connected to the free amine group in 12b, leading to the complete structure embodied by formula III:
12b+“R1”→ III

The introduction of R1, the nature of which is as described on page 3 for the general formula III, is achieved by the use of standard reactions that are well known to the person skilled in the art, such as alkylation, acylation, sulfonylation, formation of ureas, etc.

Formula 12c describes an N-protected variant of compound 12b. Group “PG” in formula 12c represents a protecting group of the type commonly utilized by the person skilled in the art. Therefore, in a second phase of the synthesis, group PG is cleaved to transform compound 12c into compound 12b. Compound 12b is subsequently advanced to structures of formula I as detailed above.

Formula 12d describes a nitro analogue of compound 12b. In a second phase of the synthesis, the nitro group of compound 12d is reduced by any of the several methods utilized by the person skilled in the art to produce the corresponding amino group, namely compound 12b. Compound 12b thus obtained is subsequently advanced to structures of formula III as detailed above.

Examples of compound synthesis is found in our previous applications WO 2004/014903 and U.S. 60/513,214, incorporated herein by reference.

The expression plasmodium related diseases or infections as referred herein includes malaria and all forms of nematodes or parasitis related diseases or infections in human or animal including shistosoma mansoni, Brugia malayi, onchocerca volvulus.

In a further embodiment, c-kit inhibitors as mentioned above are inhibitors of wild type or mutant activated c-kit. In this regard, the invention contemplates a method for treating plasmodium or nematodes related diseases or infections comprising administering to a human in need of such treatment a compound that is a selective, potent and non toxic inhibitor of c-kit obtainable by a screening method which comprises:

a) bringing into contact (i) activated c-kit and (ii) at least one compound to be tested; under conditions allowing the components (i) and (ii) to form a complex,

b) selecting compounds that inhibit activated c-kit,

c) testing and selecting a subset of compounds identified in step b), which are unable to promote death of L-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild. Alternatively, in step a) activated c-kit is SCF-activated c-kit wild.

A best mode for practicing this method consists of testing putative inhibitors at a concentration above 10 μM in step a). In step c), IL-3 is preferably present in the culture media of IL-3 dependent cells at a concentration comprised between 0.5 and 10 ng/ml, preferably between 1 to 5 ng/ml. These screening may be performed following our previous application WO 03/003006, which is incorporated herein by reference.

Therefore, the invention embraces the use of the compounds defined above to manufacture a medicament for treating, preventing or delaying plasmodium infections such as malaria and all forms of nematodes or parasitis related diseases or infections in human or animal including shistosoma mansoni, Brugia malayi, onchocerca volvulus.

The pharmaceutical compositions utilized in this invention may be administered by any number of routes including, but not limited to, oral, intravenous, intramuscular, intraarterial, intramedullary, intrathecal, intraventricular, transdermal, subcutaneous, intraperitoneal, intranasal, enteral, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

More particularly, the invention relates to a pharmaceutical composition intended for oral administration.

Pharmaceutical compositions suitable for use in the invention include compositions wherein compounds for depleting mast cells, such as c-kit inhibitors, or compounds inhibiting mast cells degranulation are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred.

### Example 1

### AB compounds of Formula III, IV, V and X are Selective and Potent c-Kit and Mast Cell Inhibitors

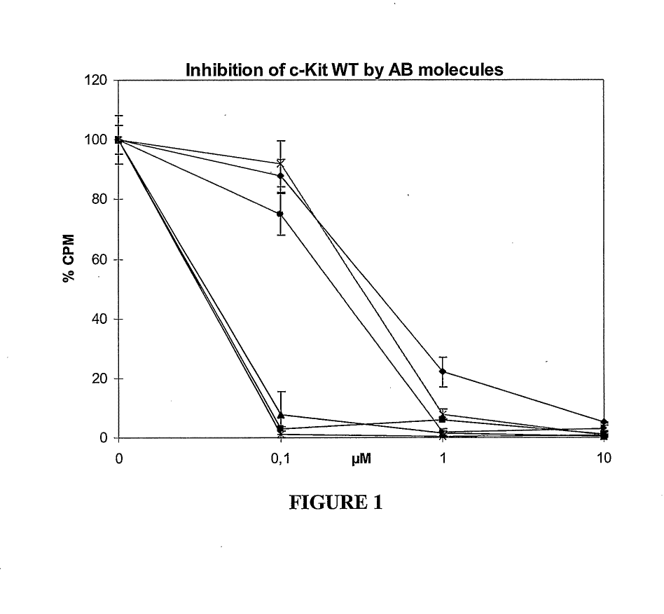

The specific compounds as listed above are non limitative illustrative examples of AB compounds. They display IC50 below 5 μM, 1 μM or even 0.1 μM on different forms of c-KIT (FIG. 1). Also, these AB compounds are selective for c-KIT versus other tyrosine kinases (Table 1).

TABLE 1
Inhibition of various protein tyrosine
kinases by the AB compound in vitro

| Enzyme | Cell line |
|---|---|
| In vitro enzymatic assay on purified kinases | IC50 [μM] |
| c-Kit | 0.01 |
| PDGF-beta | 0.49 |
| ABL1 | 5.7 |
| VEGFR1 | IC50 > 100 |
| EGFR | IC50 > 100 |
| FGFR1 | IC50 > 100 |
| FLT3 | IC50 > 100 |
| JAK2 | IC50 > 100 |
| AKT1 | 57 |
| PKC-alpha | 100 |
| SRC | IC50 > 100 |
| IGF1R | IC50 > 100 |
| PIM1 | 19 |

In addition, the AB compounds potently and dose-dependently inhibited the growth of the mast cells (MC) when they were cultured in the presence of SCF (with an IC50 of <0.1 μM). Again these in vitro data confirmed the potent and selective inhibitory activity of c-Kit tyrosine kinase activity as well as the ability of the AB compound to inhibit almost completely the survival of MC population at concentration lower than 0.1 μM. AB compounds have also been shown to deplete mast cells in vivo. The AB compound has successfully completed preclinical development in September 2003. Safety pharmacology studies revealed no significant effects of the AB compound on the central nervous, cardiovascular and respiratory systems.

1. A method for treating and/or preventing or delaying infections with plasmodium or nematodes comprising administering a compound capable of depleting mast cells or a compound inhibiting mast cells degranulation in a human in need of such treatment.

2. The method according to claim 1 for treating patients suffering from plasmodium or nematodes infections comprising administering a c-kit inhibitor to a human in need of such treatment.

3. The method according to claim 2, wherein said c-kit inhibitor is a non-toxic, selective and potent c-kit inhibitor wherein it is unable to promote death of IL-3 dependent cells cultured in presence of IL-3.

4. The method according to claim 1 wherein said inhibitor is selected from the group consisting of:
2-(3-Substitutedaryl)amino-4-aryl-thiazoles such as 2-(3-amino)arylamino-4-aryl-thiazoles,
2-aminoaryloxazoles,
pyrimidine derivatives, more particularly N-phenyl-2-pyrimidine-amine derivatives,
indolinone derivatives, more particularly pyrrol-substituted indolinones,
monocyclic, bicyclic aryl and heteroaryl compounds,
and quinazoline derivatives.

5. The method according to claim 4, wherein said c-kit inhibitor is selected from compounds belonging to the 2-(3-Substitutedaryl)amino-4-aryl-thiazoles of formula III: 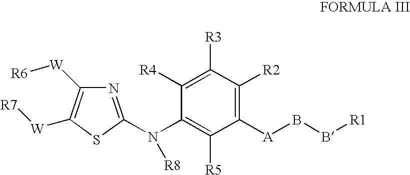
wherein
R⁶ and R⁷ are independently from each other chosen from one of the following:
i) hydrogen, a halogen (selected from F, Cl, Br or I),
ii) an alkyl¹ group defined as a linear, branched or cycloalkyl group containing from 1 to 10 carbon atoms, or from 2 or 3 to 10 carbon atoms, (for example methyl, ethyl, propyl, butyl, pentyl, hexyl . . . ) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl;
(iii) an aryl¹ group defined as phenyl or a substituted variant thereof bearing any combination, at any one ring position, of one or more substituents such as
halogen(selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
(v) trifluoromethyl, carboxyl, cyano, nitro, formyl, hydroxy, N(alkyl¹)(alkyl¹), and amino, the latter nitrogen substituents optionally in the form of a basic nitrogen functionality,
R⁸ is one of the following:
(i) hydrogen, or
(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
(iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality,
R2, R3, R4 and R5 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or D, oxygen, and nitrogen, the latter optionally in the form of a pendant basic nitrogen functionality,
A is: CH2, O, S, SO2, CO, or COO,
B is a bond or NH, NCH3, NR*, (CH2)n (n is 0, 1 or 2), O, S, SO2, CO, or COO,
B′ is a bond or NH, NCH3, NR*, (CH2)n (n is 0, 1 or 2), O, S, SO2, CO or COO;
R* being an alkyl¹, aryl¹ or heteroaryl¹
W is a bond or a linker selected from NH, NHCO, NHCOO, NHCONH, NHSO2, NHSO2NH, CO, CONH, COO, COCH2, (CH2)n (n is 0, 1 or 2), CH2-CO, CH2COO, CH2-NH, O, OCH2, S, SO2, and SO2NH,
R¹ is:
a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality
c) an alkyl¹, aryl¹ or heteroaryl¹.

6. A method according to claim 5, wherein said c-kit inhibitor is selected from compounds of formula V: 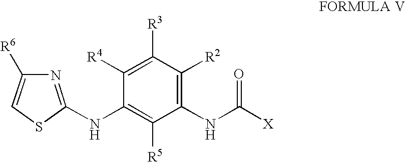
wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

7. The method according to claim 4, wherein said c-kit inhibitor is selected from 2-aminoaryloxazoles of formula X: 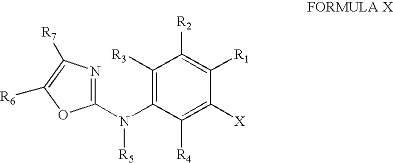
wherein substituents R1-R7 and X are defined as follows:
R1, R2, R3 and R4 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.
R5 is one of the following:
(i) hydrogen, or
(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
(iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆-alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2—R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality,
R6 and R7 each independently are selected from:
i) hydrogen, a halogen (selected from F, Cl, Br or I), or
ii) an alkyl¹ group defined as a linear, branched or cycloalkyl group containing from 1 to 10 carbon atoms and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality; as well as a cycloalkyl or aryl or heteroaryl group optionally substituted by a a pendant basic nitrogen functionality, or
(iii) an aryl¹ group defined as phenyl or a substituted variant thereof bearing any combination, at any one ring position, of one or more substituents such as
halogen(selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, or
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2—R or NHSO2NH—R or CO—R or COO—R or CONH—R or SO2-R or SO2NH—R wherein R corresponds to hydrogen, alkyl¹, or
(v) an O-aryl¹, or NH-aryl¹, or O-heteroaryl¹ or NH-heteroaryl¹ group
(vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or
(vi) NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl,
X is:
—NR9R10, wherein R9 and/or R10 are hydrogen or:
i) an alkyl¹ group, CF3 or
ii) an aryl¹, heteroaryl¹ or cycloalkyl group optionally substituted by a a pendant basic nitrogen functionality, or
iii) a CO—R, COO—R, CON—RR′ or SO2-R, where R and R′ are a hydrogen, alkyl¹, aryl¹ or heteroaryl¹, optionally substituted by a a pendant basic nitrogen functionality; or:
—CO—NR9R10, wherein R9 and/or R10 are hydrogen or:
i) an alkyl¹ group, CF3 or
ii) an aryl¹, heteroaryl¹ or cycloalkyl group optionally substituted by a a pendant basic nitrogen functionality.

8. The method according to claim 4, wherein said inhibitor is selected from the group consisting of N-phenyl-2-pyrimidine-amine derivatives having the formula II: 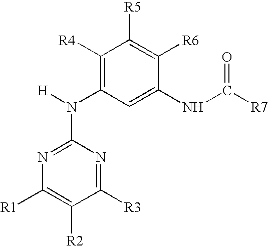
wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

9. The method according to claim 8, wherein said inhibitor is the 4-(4-méhylpipérazine-1-ylméthyl)-N-[4-méthyl-3-(4-pyridine-3-yl)pyrimidine-2ylamino)phényl]-benzamide.

10. A method for treating plasmodium or nematodes related diseases or infections comprising administering to a human in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

11. The method according to claim 1 for treating, preventing or delaying plasmodium infections such as malaria and all forms of nematodes or parasitis related diseases or infections in human or animal including shistosoma mansoni, Brugia malayi, onchocerca volvulus.

12. A compound as defined in medicament for treating, preventing or delaying plasmodium infections such as malaria and all forms of nematodes or parasitis related diseases or infections in human or animal including shistosoma mansoni, Brugia malayi, onchocerca volvulus, comprising an effective amount of a compound as claimed in claim 1 and a pharmaceutically acceptable carrier.
